(19) United States
(12) Patent Application Publication
(10) Pub. No.: US 2004/0152976 A1
(43) Pub. Date: Aug. 5, 2004

(54) Device for examining tissue using light

(76) Inventors: Arne Hengerer, Erlangen (DE); Thomas Mertelmeier, Erlangen (DE)

Correspondence Address: HARNESS, DICKEY & PIERCE, P.L.C., P.O.BOX 8910, RESTON, VA, 20195, US

(21) Appl. No.: 10/469,280

(22) PCT Filed: Feb. 21, 2002
(86) PCT No.: PCT/DE02/00638

(30) Foreign Application Priority Data
Feb. 28, 2001 (DE) 101 09 539.2

(51) Int. Cl. A61B 6/00

(52) U.S. Cl. 600/431; 600/473; 600/476; 250/458.1

(57) ABSTRACT

The invention relates to a device for examining tissue using light and involving the use of a fluorescent contrast medium. Said device comprises a hand-held applicator (1) with an application surface (3), which contains at least one light exit zone (8) for excitation light and at least one light entry zone (9) for fluorescent light. Said zones are arranged in such a manner that the direction of detection of the fluorescent light is counter to the direction of the primary diffusion of the excitation light.

The invention relates to a device for examining tissue using light, having

a) a photoemission unit with at least one light source emitting light, and with at least one light exit zone for emitted light per light source,

b) a photodetection unit with at least one light detector, which delivers output signals corresponding to the detected light, at least one light entry zone for fluorescent light being provided per light detector,

c) a hand-held applicator which contains at least the light exit zone(s) and at least the light entry zone(s), which are arranged in such a way that the detection direction of the received light is directed counter to the principal propagation direction of the excitation light, and

d) an evaluation unit which determines the intensity of the received fluorescent light from the output signal of the photodetector.

Since, in such known devices, the light exit and light entry zones are arranged in a hand-held applicator, the device has to function in reflection operation, that is to say the detection direction of the received light is directed opposite to the principal propagation direction of the emitted light.

Since the light which is used does not penetrate very far into tissue, in transmission operation in which the light exit and light entry zones are arranged facing one another on opposite sides of the tissue to be examined, it is only possible to examine relatively thin body sections which are accessible from both sides. With a device functioning in reflection operation, however, all the tissue parts near the surface can be examined (for example mamma, thyroid, skin, and even body cavities endoscopically). However, optical imaging with intrinsic contrast has a very low sensitivity because of the light scattering and the relatively small penetration depth. Especially in reflection operation, the sensitivity for deeper-lying structures is relatively low.

Devices with a hand-held applicator would be desirable for examinations of the female breast (mamma diagnosis) since, for example, the patient's mamma to be examined could then be scanned by means of the applicator while the patient is in the supine position, in a way which is convenient for the patient as well as the medical staff, similarly as in an ultrasound examination. However, there is the risk that the particularly relevant mamma regions near the ribs may lie too deep to be still examinable reliably.

DE 198 54 292 DE C2 discloses devices for examining tissue using light, in which the photodetection unit receives not back-scattered or transmitted fractions of the emitted light, but instead fluorescent light which comes, for example, from one of the body's own enzymes which is stimulated in fluorescence by means of the light coming from the photoemission unit.

Such devices are furthermore known in which the fluorescent light comes not from the body's own substances but instead from a fluorescent contrast agent introduced into the tissue, which is stimulated in fluorescence by means of the light coming from the photoemission unit. In this case, use is made of the fact that particular fluorescent contrast agents which, for example, can be produced using molecular biological methods, concentrate in tumors. Fluorescent contrast agents in the context of the invention contain a fluorescent dye which is coupled to a carrier molecule, for example a peptide.

In both cases, the fluorescent light only needs to cover the distance between the point of origin and the detector, which is in general only about half the distance when using intrinsic contrast. The scattering and absorption are therefore less, which leads to a further increase in the sensitivity and the position resolution.

It is therefore an object of the invention to refine a device of the type mentioned in the introduction so that it has an improved sensitivity, despite the use of a hand-held applicator.

According to the invention, this object is achieved by the features of patent claim 1.

Accordingly, the device is configured for use with a fluorescent contrast agent such that the photoemission unit emits excitation light, which comprises light with a wavelength that stimulates the respective contrast agent to emit fluorescent light with a fluorescence wavelength, and the photodetection unit is designed so that it detects the fluorescent light and delivers output signals corresponding to the detected light.

In spite of a hand-held applicator which contains both the light exit zones and the light entry zones, the device according to the invention then has improved sensitivity.

It is hence clear that the disadvantages of a hand-held applicator containing both the light exit zones and the light entry zones can surprisingly be avoided by using a contrast agent. In this case it is essential that, owing to the use of a contrast agent and despite the arrangement of the light exit zones as well as the light entry zones in a hand-held applicator, the device according to the invention only seems to function in reflection operation since it is not the back-scattered fractions of the light introduced by means of the photoemission unit which is detected, but instead fluorescent light.

Unlike the back-scattered light fractions detected in true reflection operation, which contain only very little position information, the position information contained in the detected fluorescent light is incomparably greater, at least in the event that there is a tumor, since the point of origin of the detected fluorescent light is essentially restricted to the tumor owing to the concentration of the contrast agent in the tumor.

In a preferred embodiment of the invention, with a view to fast examination process, larger areas can be examined simultaneously since the photodetection unit has a plurality of matricially arranged light entry zones, and according to a further preferred embodiment of the invention, a light exit zone is assigned to each light entry zone with a view to increased sensitivity.

In variants of the invention, the applicator contains the light sources of the photoemission unit and/or the light detectors of the photodetection unit. This is advantageous especially if the light sources and/or light detectors have a small overall size, which is true for example if laser diodes are being used as light sources and photodiodes, for example avalanche photodiodes, are being used as light detectors.

Provision may, however, also be made for the light sources and/or the light detectors not to be contained in the applicator, in which case suitable light guide means, for example optical fibers, must connect the light output zones and the light input zones respectively to the light sources and the light detectors. Especially in this embodiment, CCDs are also suitable as detectors.

In a particularly preferred embodiment of the invention, the light exit zones and light entry zones are arranged in an at least substantially square application face, whose side lengths are dimensioned in such a way that, for a fraction of more than 75% of all women, a mamma of a woman occupying a supine position is fully detectable by four at least mutually adjacent positions of the application face, which form an at least substantially square arrangement, which is the case when the application face has a side length of 80 mm. The vast majority of all patients can then be examined quickly since just four positions of the application face are sufficient to record the data required for representing the mamma; these positions may mutually overlap in the case of smaller mammae.

In the case of patients with very large mammae, more than four positions of the application face are needed in order to scan a mamma fully, for example nine positions.

According to a variant of the invention, the device may comprise means for delivering the contrast agent, for example a contrast agent injector. Provision may, however, also be made for the contrast agent injection to be carried out manually by medical staff.

An exemplary embodiment of the invention is represented in the appended schematic drawings, in which:

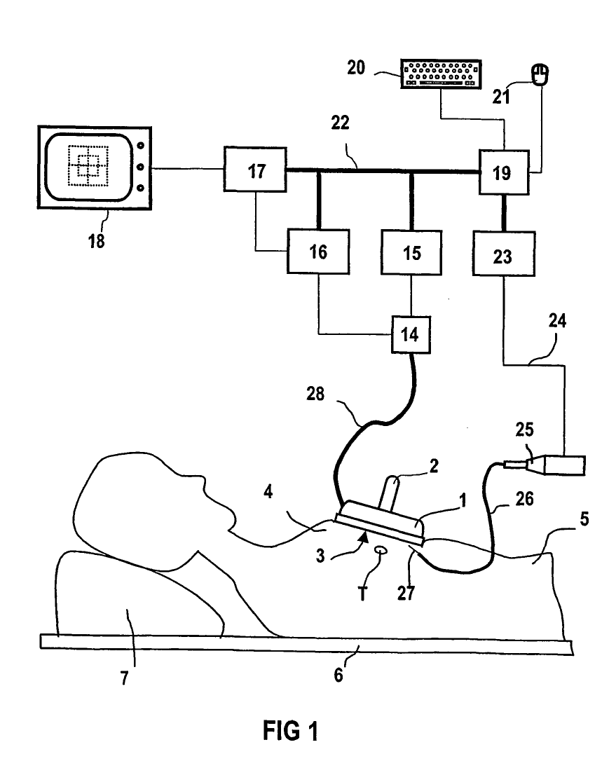

FIG. 1 shows a device according to the invention in a partially block-diagrammatic representation,

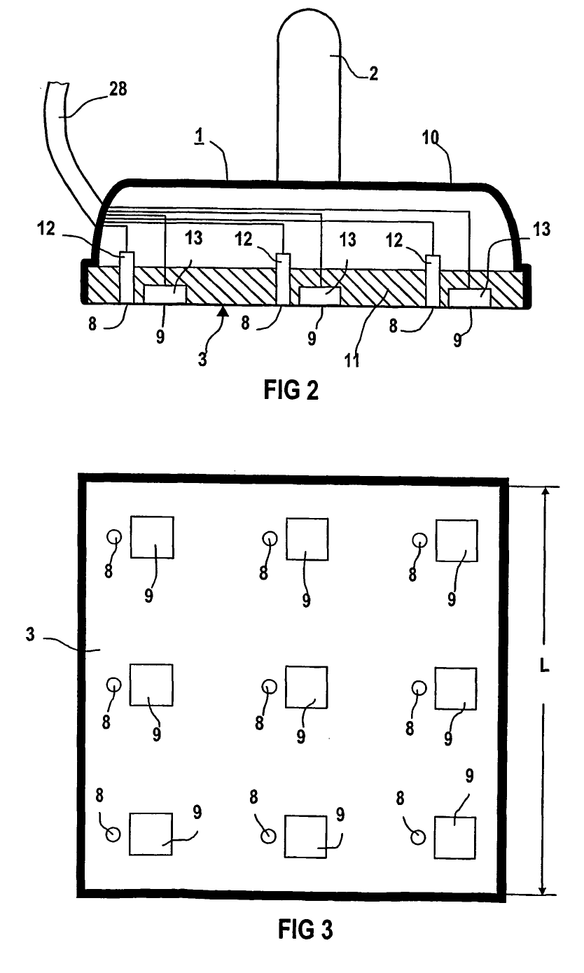

FIG. 2 shows a section through the applicator of the device according to FIG. 1, in an enlarged representation,

FIG. 3 shows a view of the application face of the applicator according to FIG. 1, in a likewise enlarged representation, and

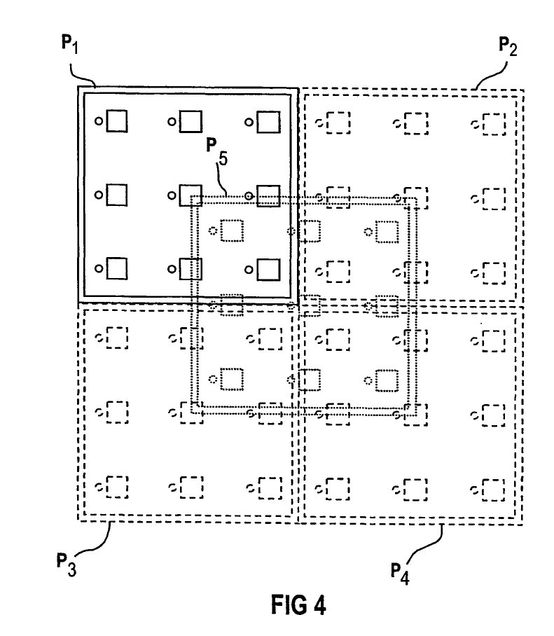

FIG. 4 shows a diagram illustrating the use of the device according to the invention.

As can be seen from FIG. 1, the device according to the invention has a hand-held applicator 1 which is provided with a handle 2 for easier manipulation. The applicator 1 has an application face 3, which is intended to be brought in engagement with the body surface of a patient, a female patient 5 in the case of the exemplary embodiment which is illustrated, in order to examine tissue located below the application face 3 by using the light, an optical coupling fluid (immersion oil) preferably being applied between the application face 3 and the patient's body surface.

In the case of FIG. 1, an application from mamma diagnosis is represented, i.e. the applicator 1 is brought in engagement via its application face 3 with the mamma 4 of the patient 5, who is lying in a supine position on a table 6 with a headrest 7. In order to bring the mamma 4 into a favorable position for the examination, it may be expedient for the patient 5 to place her hands between the back of her head and the headrest 5 when the examination is being carried out.

In the vicinity of the application face 3, as can be seen from FIGS. 2 and 3, light exit zones 8 and light entry zones 9 are arranged which are in engagement with the body surface of the patient 5 when an examination is being carried out, so that light can be sent into the patient's mamma 4 through the light exit zones and light emerging from the mamma 4 of the patient 5 can be received by means of the light entry zones 9.

In the case of the exemplary embodiment which is described, the application face 3 is of square configuration, nine light exit zones 8 and nine light entry zones 9 being provided in each case, and being respectively arranged in a square 3×3 matrix. In this case, the arrangement is organized so that a light exit zone 8 and a light entry zone 9 are in each case arranged close to one another.

As can be seen from FIG. 2, the applicator 1 has a key-shaped housing 10 with the handle 2, the key-shaped housing 10 being closed by a support plate 11 the outside of which forms the application face 3.

In the case of the invention, the examination of the mamma 4 is carried out after delivery of a fluorescent contrast agent which concentrates in tumors, for example in the tumor T contained in the mamma 4, and/or is activated therein.

In order to deliver such a contrast agent, for example cyanine fluorescent dyes which are coupled to tumor-specific peptides, proteins or antibodies, a contrast agent injector 25 actuated by a drive circuit 23 via a line 24 is provided, which delivers the contrast agent to the patient 5 in a dosed fashion via a tube 26 with a cannula 27.

In the case of the exemplary embodiment which is described, laser diodes 12 whose light exits form the light exit zones 8 are arranged as light sources in the support plate 11. Photodiodes 13, whose light entries form the light entry zones 13, are furthermore accommodated as light detectors in the support plate 11.

The laser diodes 12 are connected by corresponding wires of a cable 28 via an interface unit 14 to a supply circuit 15, which supplies the laser diodes 12 with a current such that the laser diodes 12 emit light via the exit zones 8, referred to below as excitation light, which has a wavelength such that it stimulates the contrast agent administered to the patient 5 in fluorescence.

The photodiodes 13 are connected via corresponding lines of the cable 28 and the interface unit 14 to a detector circuit 16, which detects the output signals delivered by the photodiodes 13. The photodiodes 13 are designed so that, if necessary with the aid of filter means (not shown) arranged in the beam path between the light exit zone and the photodiode, they essentially respond only to the light coming from the contrast agent, which is referred to below as fluorescent light.

It is hence clear that the light exit zones 8 and the light entry zones 9 in the applicator 1 are arranged in such a way that the detection direction of the fluorescent light is directed counter to the principal propagation direction of the excitation light.

The signals delivered by the detector circuit 16 proceed to an evaluation unit 17 which generates an image from them and displays it on a monitor, in a manner which will be described in further detail.

A control unit 19, to which a keyboard 20 and a further operating element, a mouse 21 in the case of the exemplary embodiment which is described, are connected, is used to control the device and is connected for this purpose to the supply circuit 15, the detector circuit 16, the evaluation unit 17 and the drive circuit 23 of the contrast agent injector 25 via a control bus 22. The measuring arrangement is operated by means of the keyboard 20 and the mouse 21, for example with the aid of user menus which are displayed on the monitor 18.

In the case of the exemplary embodiment which is represented, the application face 3 has an edge length of L=80 mm.

This ensures that, for a fraction of more than 75% of all women, a mamma of the woman lying in the supine position can be fully detected for the purpose of the examination by carrying out examinations for four positions P₁ to P₄ of the applicator 1, for which the application faces 3 form an at least substantially square arrangement and, depending on the size of the mamma, are adjacent to one another or mutually overlap as illustrated in FIG. 4.

The current position P₁ of the applicator 1 is represented by solid lines in FIG. 4, while the other positions P₂ to P₄ which the applicator 1 must occupy for complete scanning of the mamma 4 are indicated by dashed lines.

If extra information about the central region of the mamma 4 is important, a further examination may be carried out for a central position P₅ of the applicator 1, which is indicated by dots in FIG. 4.

Once the contrast agent has been administered to the patient 5, then after a time has elapsed which is sufficient for the contrast agent to concentrate in any tumor T which may be present, the applicator 1 is brought in engagement with the mamma 4 of the patient 5 via its application face 3. An online image corresponding to the intensities of the fluorescent light detected by means of the individual photodiodes 13 then appears on the monitor 18, which image is determined by the evaluation unit 17 and by means of which image a first orientation and overview is made possible.

The pixels of the images determined by the evaluation unit 17 correspond to the light entry faces 9 in terms of arrangement and number, a gray value corresponding to the respective intensity of the detected by means of fluorescent light being assigned to the individual pixels according to an intensity/gray value scale.

After orientation has taken place, images are recorded for the positions P₁ to P₄ and optionally the position P₅, and the data thereby obtained corresponding to the intensities of the fluorescent light detected by means of the individual photodiodes 13 are stored in the evaluation unit 17 if so required. From these data, the evaluation unit 17 determines images in the described manner and stores them if so required.

The images obtained for the positions P₁ to P₄ and optionally the position P₅ are displayed by the evaluation unit 17 on the monitor 18 as indicated in FIG. 1, according to the positions P₁ to P₄ or P₅, the opportunity being available to modify the position of the images relative to one another, for example by means of the mouse 21, so that the positions of the images correspond at least approximately to the positions of the applicator. The individual images may also be combined to form an overall image by means of a software program.

In a first operating mode, the device according to the invention may be operated in such a way that light emerges simultaneously from all the light exit zones 8 and light is simultaneously detected over all the light entry zones 9.

In alternative operating modes, the opportunity is available to activate the light exit zones 8 sequentially and to detect over all the light entry zones 9 or a subset of the light entry zones 9, for example the four light entry zones 9 respectively lying closest to the activated light exit zone 8.

Lastly, the opportunity is available in a further operating mode to sequentially activate only one of the light exit zones 8 in each case, and to detect by means of the light entry zone 9 assigned to it.

The number and arrangement of the light exit zones and light entry zones present in the case of the exemplary embodiment which is described should only be taken by way of example. The same applies to the type of light sources and light detectors provided in the case of the exemplary embodiment. For example, photomultipliers may also be used as the light detectors.

The dimensions of the application face provided in the case of the exemplary embodiment which is described, and the positions of the application face to be selected for carrying out an examination, are also to be taken only by way of example.

In the case of the exemplary embodiment which is described, the examination is carried out in the supine position, which is usually favorable. In order to examine special regions of the mamma, however, it may be expedient to reposition the patient.

The contrast agent injector provided in the case of the exemplary embodiment which is described may also be obviated if the contrast agent is injected manually into the patient.

In the case of the exemplary embodiment which is described, the device according to the invention is used for the purposes of mamma diagnosis. The device according to the invention may, however, also be used for other diagnostic purposes, for example the examination of (axial) lymph nodes or to investigate non-oncological problems; care should be taken that the tissue section to be penetrated by the excitation light as well as the fluorescent light during the examination ought not to be substantially thicker than 50 mm.

The device according to the invention offers the advantage of not being dangerous since neither radioactive radiation nor X-rays are used, and it can also be constructed as an inexpensive device which is easy to handle.

The opportunity furthermore exists to superimpose images obtained by means of the device according to the invention with images that have been obtained by other modalities; for example, images obtained by using the device according to the invention may be superimposed with X-ray mammography images.

In the future, a further increase of the sensitivity achievable in examinations using a device according to the invention is to be expected, specifically through new contrast agents produced using modern molecular biological methods.

1. A device for examining tissue using light with the aid of a fluorescent contrast agent, having
a) a photoemission unit with at least one light source which emits excitation light, which comprises light with a wavelength that stimulates the respective contrast agent to emit fluorescent light with a fluorescence wavelength, and with at least one light exit zone for the excitation light per light source,
b) a photodetection unit with at least one light detector for detecting fluorescent light, which delivers output signals corresponding to the detected light, at least one light entry zone for fluorescent light being provided per light detector,
c) a hand-held applicator which contains at least the light exit zone(s) and at least the light entry zone(s), which are arranged in such a way that the detection direction of the fluorescent light is directed counter to the principal propagation direction of the excitation light, and
d) an evaluation unit which determines the intensity of the received fluorescent light from the output signal of the photodetector.

2. The device as claimed in claim 1, whose photodetector unit comprises a plurality of matricially arranged light entry zones.

3. The device as claimed in claim 1 or 2, in which a light exit zone is assigned to each light entry zone.

4. The device as claimed in one of claims 1 to 3, whose applicator contains the light source(s) of the photoemission unit.

5. The device as claimed in one of claims 1 to 4, whose applicator contains the light detector(s) of the photodetection unit.

6. The device as claimed in one of claims 1 to 5, whose light exit zone(s) and light entry zone(s) are arranged in an at least substantially square application face, whose side lengths are dimensioned in such a way that, for a fraction of more than 75% of all women, a mamma of a woman occupying a supine position is fully detectable by four at least mutually adjacent positions of the application face, which form an at least substantially square arrangement.

7. The device as claimed in claim 6, whose application face has a side length of 80 mm.

8. The device as claimed in one of claims 1 to 7, which has means for delivering the contrast agent.

9. A method for operating a device as claimed in one of claims 1 to 8, in which an optical coupling fluid is arranged between the light source and light detector, on the one hand, and the surface of the tissue to be examined, on the other hand.
